(19) United States
(12) Patent Application Publication
(10) Pub. No.: US 2015/0202354 A1
(43) Pub. Date: Jul. 23, 2015

(54) Wound Dressing

(71) Applicant: Integrated Healing Technologies, LLC, Franklin, TN (US)

(72) Inventor: Billy Russell Wall, Arrington, TN (US)

(73) Assignee: Integrated Healing Technolgies, LLC, Franklin, TN (US)

(21) Appl. No.: 14/425,570

(22) PCT Filed: Sep. 4, 2013
(86) PCT No.: PCT/US2013/058062

§ 371 Date: Mar. 3, 2015

Related U.S. Application Data
(60) Provisional application No. 61/696,660, filed on Sep. 4, 2012.

(51) Int. Cl. A61M 1/00; A61F 13/02

(52) U.S. Cl. CPC A61M 1/0088; A61F 13/0206; A61F 13/022

(57) ABSTRACT

The wound dressing combines an integrated mechanical aspirator for drawing exudate and fluids from the wound site and an internal absorbent pad for collecting exudate and fluids within the wound dressing. The collection pad has multi-layers including a layer of hyper-absorbent material, which acts as the fluid collection medium. The aspirator is a type of simple bulb syringe, which is integrated into top of the wound dressing in fluid communication with the collection pad. The aspirator has a hollow bulb head and a one-way purge vent. The vent allows air to be purged from the bulb head when manually compressed. Once compressed, the structural and material resilience of the bulb head causes its walls to expand back to their original shape thereby creating a vacuum (negative) pressure within the bulb head that assists in drawing exudate and fluids from the wound site into the collection pad.

### TECHNICAL FIELD

This invention relates to a wound dressing, and in particular a wound dressing including an integrated aspirator and exudate collection pad.

### BACKGROUND ART

Negative pressure wound therapy has proven effective in promoting the healing of wounds. Negative pressure wound therapy involves connecting an external vacuum source to a hermetically sealed wound dressing to draw and collect exudate from the wound. While readily available in clinical settings, the apparatus for facilitating negative pressure wound therapy in the field are seldom available or convenient to use.

### DISCLOSURE OF THE INVENTION

The wound dressing of this invention combines an integrated simple mechanical aspirator for drawing exudate and other fluids from the wound site and an internal absorbent pad for collecting the exudate and other fluids within the wound dressing itself. The collection pad has multi-layers including a layer of hyper-absorbent material, which acts as the fluid collection medium. In one embodiment, the aspirator is a type of simple bulb syringe, which is integrated into the top of the wound dressing in fluid communication with the collection pad. The aspirator is formed, molded or otherwise made having a hollow bulb head and a one-way purge vent. The vent allows air to be purged from the bulb head when manually compressed. Once compressed, the structural and material resilience of the bulb head causes its walls to expand back to their original shape thereby creating a vacuum (negative) pressure within the bulb head that assists in drawing exudate and fluids from the wound site into the collection pad.

The wound dressing of this invention is ideally suited for wound treatment in operational theaters, where conventional negative pressure wound therapy apparatus and equipment are unavailable or impractical. The wound dressing does not require an external vacuum source, exudate canister or connective lines. The wound dressing can be self-employed with no tools or equipment and with little training or expertise.

The above described features and advantages, as well as others, will become more readily apparent to those of ordinary skill in the art by reference to the following detailed description and accompanying drawings.

### BRIEF DESCRIPTION OF THE DRAWINGS

The present invention may take form in various system and method components and arrangements of system and method components. The drawings are only for purposes of illustrating exemplary embodiments and are not to be construed as limiting the invention. The drawings illustrate the present invention, in which:

FIG. 1 is a perspective view of an embodiment of the wound dressing of this invention applied over a wound site;

FIG. 2 is a perspective view of the wound dressing of FIG. 1 with various segments cut away to show the layers of the dressing;

FIG. 3 is a side sectional view of the wound dressing of FIG. 1 applied over a wound site; and

FIG. 4 is a side sectional view of the wound dressing of FIG. 1 applied over a wound site showing the vacuum bulb compressed.

### BEST MODE FOR CARRYING OUT THE INVENTION

In the following detailed description of the preferred embodiment, reference is made to the accompanying drawings that form a part hereof, and in which is shown by way of illustration the specific preferred embodiment in which the invention may be practiced. The embodiment is described in sufficient detail to enable those skilled in the art to practice the invention, and it is understood that other embodiments may be utilized and that logical, structural, mechanical, electrical, and chemical changes may be made without departing from the spirit or scope of the invention. To avoid cumbersome details not necessary to enable those skilled in the art to practice the invention, the description may omit certain information known to those skilled in the art. The following detailed description is, therefore, not to be taken in a limiting sense, and the scope of the present invention is defined only by the appended claims.

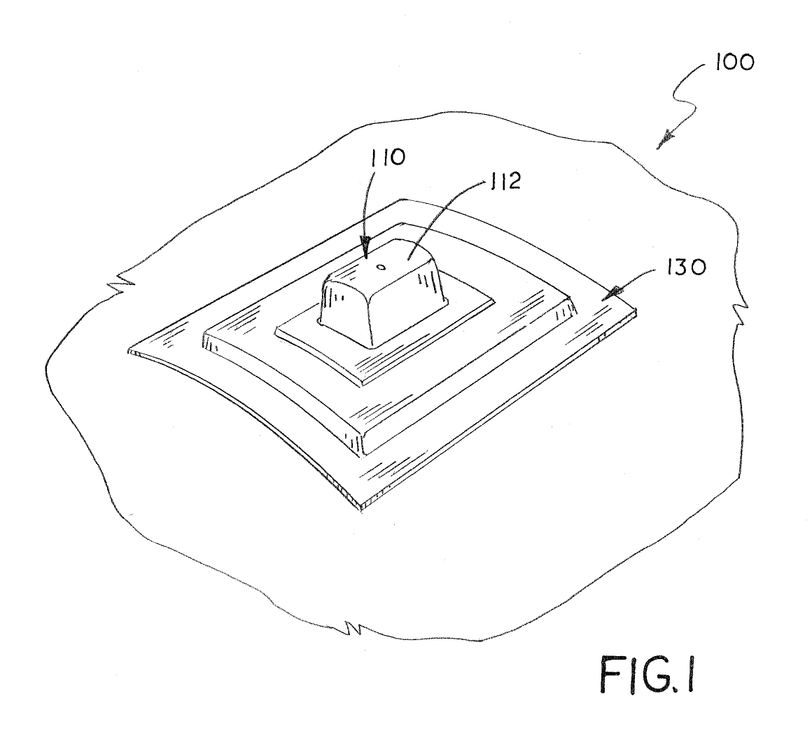

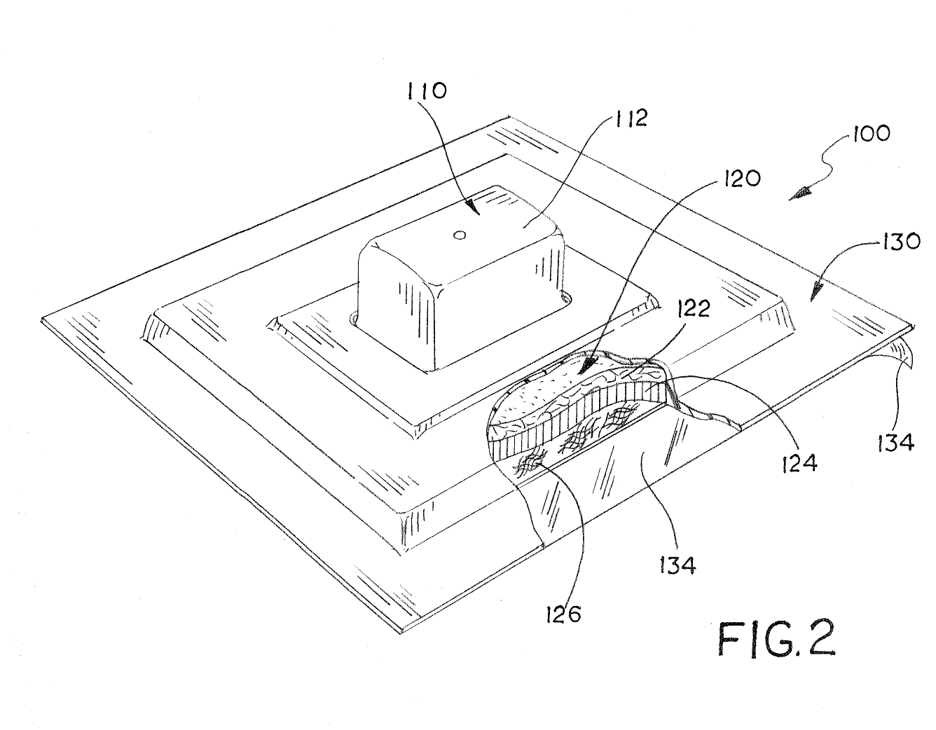

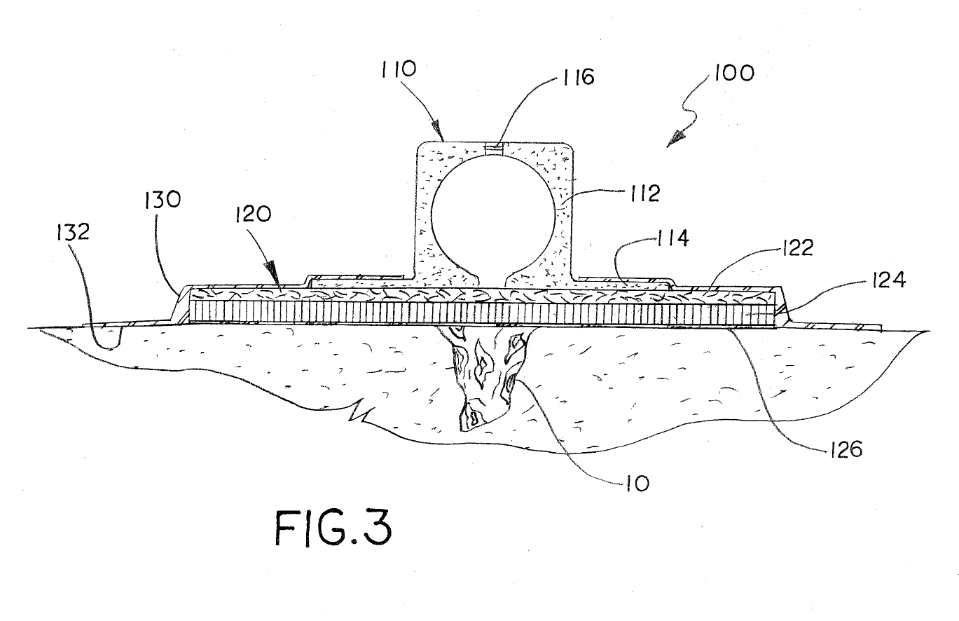

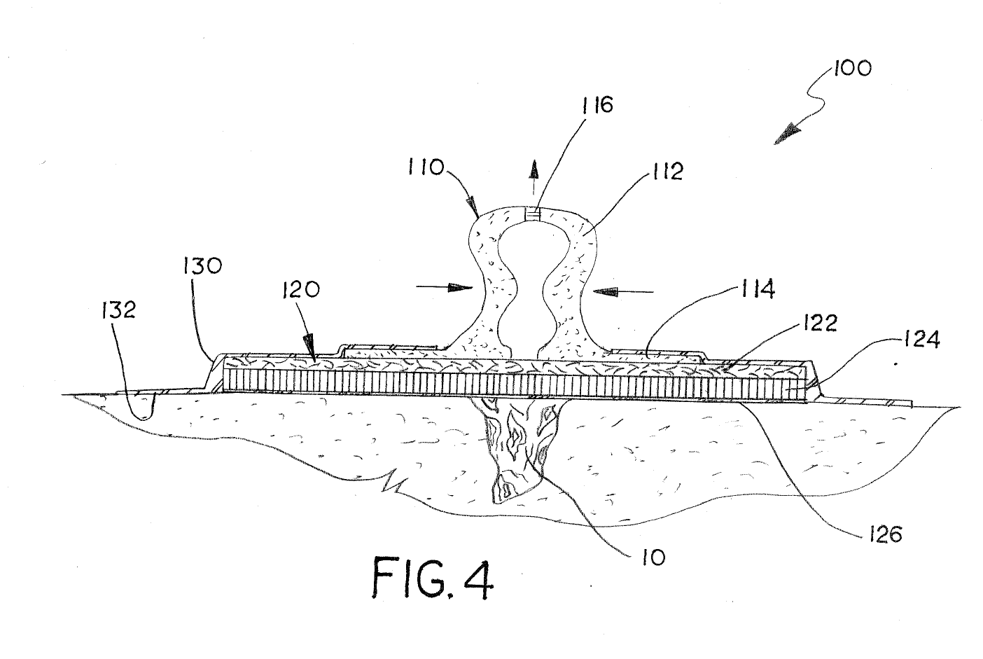

Referring now to the drawings, FIGS. 1-4 illustrate an embodiment of the wound dressing of this present invention, which is designated generally as reference numeral 100. As shown in FIG. 1, wound dressing 100 includes an integrated aspirator 110, an internal fluid collection pad 120, and a pliable drape film 150, which covers and binds the aspirator and the collection pad together.

As shown, aspirator 110 functions like a type of simple bulb syringe, which is integrated into the top of dressing 100. Aspirator 110 is formed, molded or otherwise made of a pliable sterile material suitable for medical purposes, such as a silicon rubber or Arkema Pebax® polymer and is configured to have a compressible box-shaped hollow bulb head 112 and a flat peripheral flange 114. A one-way vent 116 is formed in the top of bulb head 112, which allows air to be purged from bulb head 112 when manually compressed. Once released from compression, the structural and material resilience of the bulb head 112 causes the walls to expand back to their original shape creating the suction, that is a vacuum (negative) pressure within the bulb head, which assists in drawing exudate and fluids from wound site 10 into collection pad 120. It should be noted that in other embodiments of this invention the integrated aspirator may take a variety of other mechanical forms in providing the suction that assists in drawing exudate into the dressing. By way of example only, the aspirator may take the form of a plunger type syringe or a spring loaded bulb head that expands when a mechanical tab or lever is displaced. These examples are not meant to be exhaustive and other mechanical forms of an integrated aspirator are contemplated within the teachings of this invention.

Collection pad 120 consists of a layer of top hyper-absorbent material 122, an intermediate layer of hydrophilic “white” foam 124 and a bottom layer of hydrophobic material 126. Hyper-absorbent material 122 provides the fluid collection medium for dressing 100. Typically, hyper-absorbent material 122 consists of woven or nonwoven fibers of sodium polyarylate or other similar synthetic or naturally occurring material which can absorb and retain many times its own weight in fluid volume. Alternatively, an open cell foam or other suitable material may be employed to collect and hold exudate and other fluids within dressing 100.

Hydrophilic white foam 124 acts as fluid passage from the wound site 10 to the hyper-absorbent material 122. White foam 124 is a conventional hydrophilic polyvinyl fine pore cellular foam suitable for medical uses in wound treatment. White foam 124 is selected to reduce maceration around the wound site. White foam 124 has a pore structure that allows for fluid migration in only one direction. Fluids migrate upward through the foam away from the wound site but do not migrate laterally through the foam. Exudate is pulled in one direction through foam away from the wound site and does not migrate laterally through white foam 124, thereby reducing maceration around the wound site. White foam 124 also has a surface barrier (not shown) underlying the layer of hyper-absorbent material 122 that prevents exudate and fluid absorbed by hyper-absorbent material 122 from migrating back toward the wound site over the layer of white foam.

Hydrophobic material 126 directly contacts the wound site and provides an antibacterial/antimicrobial barrier, which helps attract and bind bacteria. Hydrophobic material 126 consists of woven or non-woven, synthetic or naturally occurring, hydrophobic and therefore bacteria binding polymeric fiber material, such as that found in Sorbact® which is available from Abigo Medical AB of Askim, Sweden. The layer of hydrophobic material 110 directly overlies and contacts the tissue at the wound site, which attracts and binds bacteria from the wound within its fabric structure. It should be noted that the hydrophobic material may also be impregnated with antimicrobial active compound, to provide additional therapeutic benefits as desired.

Drape film 130 is a thin, flexible polyurethane film, which is not air or water permeable. Drape film 130 has a central opening through which the bulb head 112 extends. The bottom of drape film 130 has a high-tack, pressure sensitive adhesive coating 132. The adhesive coating adheres drape film to collection pad 120 and suction component 110. The peripheral of the bottom of drape film 130 is covered and protected by a releasable backing 134, which is used to adhere dressing 100 to the patient around the wound site. It should be noted that drape film 130 is configured to accommodate the dimensional expansion of hyper-absorbent material 122 as it swells and becomes saturated with exudate and fluid. In addition, drape film 130 is generally transparent so that hyper-absorbent material is visible through the film for monitoring the need for dressing changes.

In use, wound site 10 is cleaned and prepared before wound dressing 100 is applied. Once releasable backing 134 is removed from the periphery of drape film 130, wound dressing 100 is placed over the wound site 10. Adhesive coating 132 on the back of drape film 130 secures the dressing to the patient's skin and creates a hermetic seal around wound site 10. With wound dressing 100 in place, bulb head 112 can be manually compressed, which purges air from the interior of bulb head 112 through vent 116. When released, the structural and material resilience of the bulb head 112 causes the walls to expand back to their original shape creating the suction, that is a vacuum (negative) pressure within the bulb head, which assists in drawing exudate and fluids from wound site 2 into collection pad 120.

The wound dressing of this invention is ideally suited for wound treatment in operational theaters, where conventional negative pressure wound therapy apparatus and equipment are unavailable or impractical. The wound dressing does not require an external vacuum source, exudate canister or connective lines. The wound dressing is compact and portable and can be self-employed without tools or equipment and with little training or expertise.

The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

What is claimed is:

1. A wound dressing comprising:
a pad for collecting exudate and fluids therein;
an aspirator overlying the collection pad in communication therewith for drawing exudate and fluids from a wound site into the collection pad; and
drape means cover with the pad and aspirator for hermetically sealing the pad and aspirator over the wound site.

2. The wound dressing of claim 1 wherein the pad includes a first layer of hyper-absorbent material.

3. The wound dressing of claim 2 wherein the pad also includes a second layer of white foam underlying the first layer such that exudate and fluids migrate there through from the wound site into the first layer when the wound dressing is affixed over a wound site.

4. The wound dressing of claim 3 wherein the pad also includes a third layer of hydrophobic material underlying the second layer.

5. The wound dressing of claim 1 wherein the aspirator includes a pliable hollow bulb head in open communication with the pad and a one way vent operatively connected to the bulb head for purging air from the bulb head.

6. The wound dressing of claim 1 wherein the drape means includes a film overlying the collection pad and aspirator and adapted to adhere the skin around a wound site.

7. The wound dressing of claim 6 wherein the film has an adhesive coating on the back thereof and being adapted to be adhered to skin of the patient, and a releasable protective backing coving the adhesive coating.
